(19) United States
(12) Patent Application Publication
(10) Pub. No.: US 2017/0294608 A1
(43) Pub. Date: Oct. 12, 2017

(54) SELF-ASSEMBLED, ELECTRONICALLY-FUNCTIONAL NUCLEIC ACID NANOSTRUCTURES AND NETWORKS BASED ON THE USE OF ORTHOGONAL BASE PAIRS

(71) Applicant: THE TRUSTEES OF COLUMBIA UNIVERSITY IN THE CITY OF NEW YORK, New York, NY (US)

(72) Inventors: Simon Alexander Vecchioni, Brooklyn, NY (US); Samuel (Shalom) Jonas Wind, White Plains, NY (US)

(73) Assignee: THE TRUSTEES OF COLUMBIA UNIVERSITY IN THE CITY OF NEW YORK, NEW YORK, NY (US)

(21) Appl. No.: 15/485,016

(22) Filed: Apr. 11, 2017

Related U.S. Application Data
(60) Provisional application No. 62/321,109, filed on Apr. 11, 2016.

(51) Int. Cl. H01L 51/00; B82Y 40/00; B82Y 10/00; C12N 15/10; C12N 15/11

(52) U.S. Cl. CPC H01L 51/0093; C12N 15/102; C12N 15/11; H01L 51/0049; B82Y 10/00; B82Y 40/00; C12N 2310/3535; Y10S 977/704; Y10S 977/72; Y10S 977/796; Y10S 977/941; Y10S 977/848; Y10S 977/746; H01L 51/0558

(57) ABSTRACT

Methods and systems for engineering a nanostructure are provided. An exemplary method includes creating at least one cytosine-cytosine and/or thymine-thymine mismatch in at least one oligonucleotide sequence, placing a metal ion into the mismatch of the oligonucleotide sequence to form an electronically functionalized nanostructure, and inducing self-assembly of the oligonucleotide sequence into a defined structure.

### CROSS-REFERENCE TO RELATED APPLICATIONS

This application claims priority to U.S. Provisional Application No. 62/321,109 filed Apr. 11, 2016, the contents of which are hereby incorporated by reference in their entireties.

### NOTICE OF GOVERNMENT SUPPORT

This invention was made with government support under NNX14AM51 awarded by the National Aeronautics and Space Administration (NASA). The government has certain rights in this invention.

### BACKGROUND

The disclosed subject matter provides systems and methods for self-assembly nanoscale devices from biosynthetic materials.

Electronic circuits with reduced size and simplified construction are important to certain electronics. Nanostructures are integral to the miniaturization of the electronics. The smaller the nanostructure, the smaller the transistor—and consequently the smaller the chip. Certain nanostructures can require vast fabrication facilities and constant upkeep of machinery and resources for the top-down production of current generation devices. Certain nanostructures can be fabricated with highly complex and expensive equipment. As the fundamental limit of silicon devices is reached and lithographic methods hit the minimum scale for small circuits, there is a need to seek other alternatives.

Certain nanostructures from biosynthetic materials can have swift and reproducible assembly at low cost with a great reduction in space, resource and time allocation. For example, nanoelectronic fabrication with DNA can involve bio-degradable components, non-toxic processes, and a relatively simple fabrication process. Deoxyribonucleic acid (DNA) can self-catalyze and self-assemble into predictable structures for a variety of functions. Accordingly, nanostructure fabrication with DNA can reduce access costs to develop and repair complex nanoelectronics in relatively low-resource situations such as on the international space station, deep space missions, war zones, and non-industrial laboratories. The nanostructure can thus overcome certain limits of silicon scaling.

Thus, there remains a need for improved techniques for fabricating nanostructures with biosynthetic materials.

### SUMMARY

The disclosed subject matter provides systems and methods for self-assembly nanoscale devices from biosynthetic materials.

In certain embodiments, an exemplary method for engineering a nanostructure includes creating at least one cytosine-cytosine and/or thymine-thymine mismatch in at least one oligonucleotide sequence and placing a metal ion into the mismatch of the oligonucleotide sequence to form an electronically-functionalized nanostructure. The metal ion can be silver or mercury. In another embodiment, the oligonucleotide sequence can include at least one fluorophore and/or at least one biotin linker.

In certain embodiments, the placing further includes providing at least one metal ion to each mismatch. In another embodiment, the nanostructure can have one or more core-functionalized regions. Each of the one or more core-functionalized regions includes at least one mismatch and/or ion-binding site. The DNA nanostructure can be built through an annealing process that can have one or more cycles.

In certain embodiments, the oligonucleotide sequence can be designed by an optimization analysis. An exemplary method for the optimization analysis includes setting design constraints, initializing populations, evaluating fitness score, and iteratively selecting more fit solutions. In other embodiments, the optimization analysis can further include population dynamics, multiple populations, subpopulations, fitness tournaments, random mutation, recessive information or/and random deletion of solutions. In another embodiment, the optimization analysis can further evaluate the connectivity of multiple sequences to optimize nanostructure topology.

In certain embodiments, the nanostructure can have a continuous or discontinuous 1-atom thick chain of silver ions surrounded by the 2 nm diameter oligonucleotide helix. In another embodiment, the nanostructure can be a DNA lattice. The DNA lattice can include Holliday junction lattice and/or T-junction lattice.

### BRIEF DESCRIPTION OF THE FIGURES

The application file contains at least one drawing executed in color. Copies of this patent with color drawings will be provided by the Office upon request and payment of the necessary fee.

FIG. 1 shows a method of engineering a nanostructure, according to one exemplary embodiment of the disclosed subject matter.

FIG. 2 shows images of an exemplary embodiment of (A) a mismatch of oligonucleotides; (B) mismatch of oligonucleotides filled with a silver ion; (C) DNA duplex form without metal ion bounded; and (D) a DNA duplex with metal ion.

FIG. 3 shows an image of oligonucleotides with fluorophores.

FIG. 4 (A) shows a plot of UV spectra absorbance and (B) melting curve of silver-functionalized poly-cytosine duplexes.

FIG. 5 shows plots of COSY-H NMR bond confirmation analyses in (A) aqueous condition; (B) precipitating condition; and (C) dialyzing condition.

FIG. 6 shows electrophoresis images of (A) molar ratio; and (B) Ag+ in ligation.

FIG. 7 shows a method of optimizing the oligonucleotide sequence, according to one exemplary embodiment of the disclosed subject matter.

FIG. 8 shows a plot of an exemplary embodiment of fitness score evaluation.

FIG. 9 shows images of DNA nanostructure sequence and topology of (A) Holliday junction unit, (B) 2D lattice of Holiday junctions, (C) minimal crossover unit, and (D) various sequences weaved by the minimal crossover units, with CC mismatched denoted as S.

FIG. 10 (A) shows a DFX (five-crossover) nanostructure with silver ion binding, and (B) a close-up image of the DFX nanostructure with silver ion binding.

FIG. 11 shows an AFM micrograph image of DFX nanostructure. FIG. 12 shows an image of an exemplary embodiment of planar transistors.

FIG. 13 shows (A) A schematic illustration of two CNTs connected by DNA. (B) An AFM image of CNTs with amine linker. (C) An AFM image of CNTs with guanine linker.

### DETAILED DESCRIPTION

The disclosed subject matter provides systems and methods for self-assembly nanoscale devices from biosynthetic materials.

According to aspects of the disclosed subject matter, systems and techniques for engineering an oligonucleotide-based nanoscale device are provided. An example method can include providing orthogonal nucleic acid base pairing (i.e., departing from the standard A-T, G-C, A-U and G-U pairs) to impart electrical functionality on oligonucleotide nanostructures (e.g., structures formed using DNA, ribonucleic acid, peptide nucleic acid, locked nucleic acid, and/or xeno nucleic acid with (but not limited to) branching, crossover or sticky-end elements).

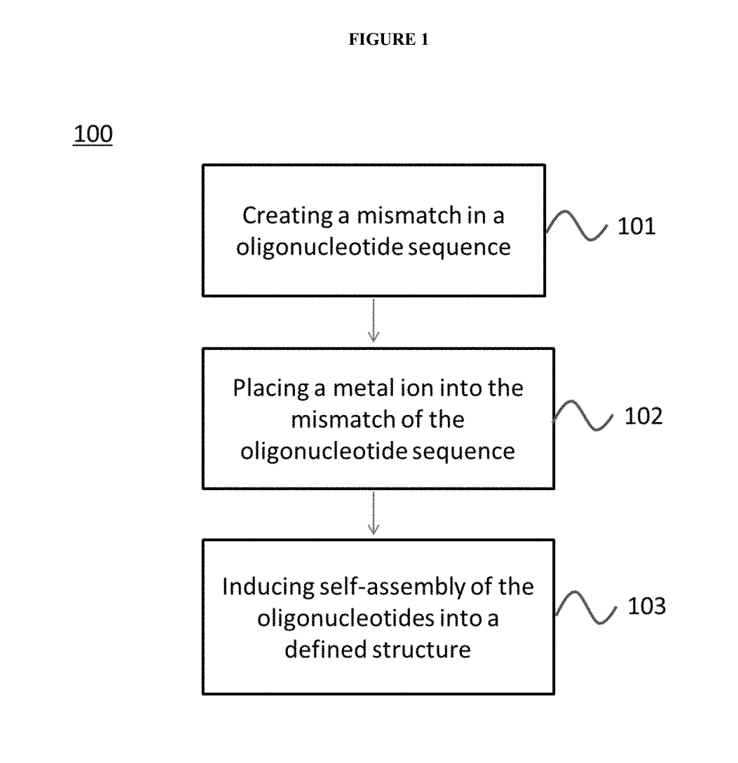

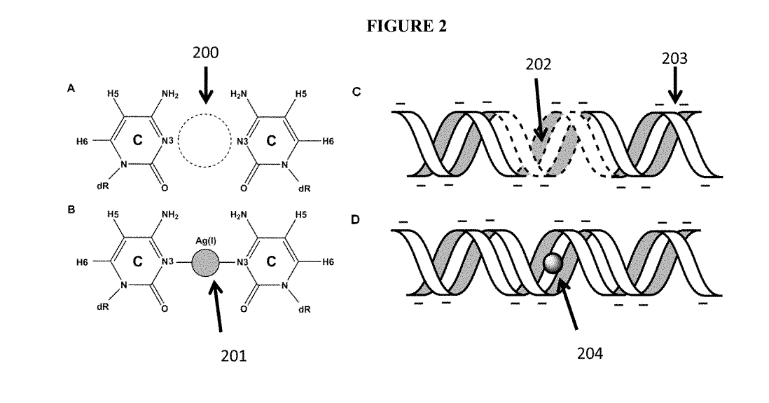

For the purpose of illustration and not limitation, FIG. 1 illustrates an exemplary method for engineering a nanostructure. In certain embodiments, a method 100 includes creating a mismatch in an oligonucleotide sequence 101. For example, at least one oligonucleotide sequence can have at least one cytosine-cytosine and/or thymine-thymine mismatch in at least one oligonucleotide sequence.

The method 100 can further include placing a metal ion into the mismatch of the oligonucleotide sequence 102. In certain embodiments, the oligonucleotide is sequence-specific and metal ions can be patterned along the oligonucleotide strand. For example, silver or mercury ions can fill the mismatches of the sequence-specific oligonucleotide strand to form an electronically-functionalized nanowire within a DNA nanostructure.

In accordance with another embodiment, the DNA (or other nucleic acid) nanostructure can be self-assembled from repeating units. The assembled nanostructure can be a one-, two-, or three-dimensional structure. The assembled structure can be either flat or non-flat. In some embodiments, the assembled nanostructure can have un-constrained growth. The assembled nanostructure can grow into the micron scale.

The method 100 can further include inducing self-assembly of the oligonucleotides into a defined nanostructure 103.

As shown in FIG. 2A, a mismatch 200 can be created by non-binding pyrimidine pairs. The mismatch or binding site can be created by standard DNA or RNA pyrimidines, as well as non-canonical nucleic acid bases such as xeno nucleic acid, peptide nucleic acid, and locked nucleic acid. In certain embodiments, a metal ion 201 such as silver or mercury can be introduced into the mismatch by providing excess ions. For example, the excess silver ions can be provided in the AgNO₃ form to the unfolded DNA strands at a ratio of 100:1 or 1:1. The oligonucleotide strands can be added to a solution comprising MOPS buffer, NaNO₃, and MgCl₂. In other embodiments, the solution can include a Trizma buffer, potassium salt, sodium salt, or other buffering agent. The buffer and the ions can be heated together in 100 uL of water at 90° C. and slowly cooled over 48 to 72 hours. In other embodiments this reaction can occur over 2 hours. In other embodiments, this reaction can be carried out at lower temperatures (e.g. 40° C., room temperature) for shorter or longer periods. In some embodiments, the ions can be supplied after annealing (heating). In other non-limiting embodiments, this reaction can be carried out at high temperature (90° C.) or lower temperature (40° C.) multiple times, and can be cycled for tens, hundreds or thousands of iterations. This reaction can involve adding new oligonucleotides or nucleic acid material not included in the primary reaction(s). In some embodiments, metal or ion species can be added in subsequent annealing reactions. The metal ions can stabilize opposing N3 sites in mismatches in short oligonucleotide sequence and DNA duplex when added to the annealing reaction at any cycle. FIG. 2C illustrates the mismatch 202 created in a DNA duplex 203. The mismatch in the DNA duplex can be filled with a metal ion 204 as shown in FIG. 2D.

In certain embodiments, the nanostructure can include double-stranded nucleic acids or/and single-stranded heterostructures including but not limited to hairpins, kissing-loops, and pseudoknots. In some embodiments, the nanostructure can be connected using sticky end attachment or/and kissing-loops. Various lengths and shapes of the nanostructure can be formed by altering the annealing and/or ligation processes by altering reactant ratios, injection times or with the use of enzymes.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

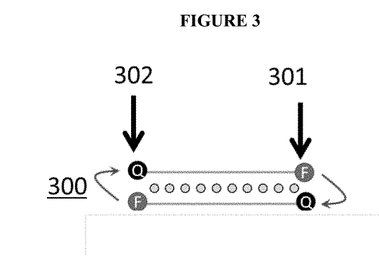

In certain embodiments, the nanostructure can include at least one fluorophore and at least one biotin linker. As silver is titrated into the environment, spatially locked DNA molecules can be folded into a duplex, causing the fluorophores to undergo resonance coupling and energy transfer (FRET) that is visible under a microscope. For example, as shown in FIG. 3A, when a double helix is formed 300, the FAM fluorophores 301 can be quenched 302. The DNA sequence with fluorophores can be used for real-time analyses of duplex formation in single molecules. The DNA sequence with fluorophore and biotin linker can be utilized to maximize and/or visualize cytosine-cytosine mismatches and silver ion placement while retaining a single robust conformation.

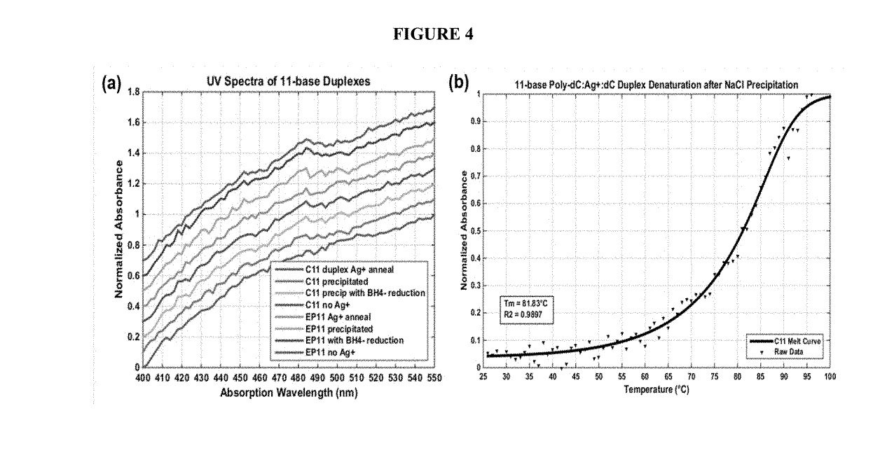

In certain embodiments, behavior of the nanostructure can be investigated using ultraviolet-visible spectroscopy (UV-Vis) and thermal denaturation. For the purpose of illustration, FIG. 4 provides UV spectra plots for chemical characterization of 11 bp, silver-incorporated poly-cytosine duplexes (dC:Ag+:dC). FIG. 4A illustrates absorbance of eight differently processed samples in the 400-500 nm range. The nanostructures are free of nanocluster contaminants. FIG. 4B illustrates a melting profile of (dC:Ag+:dC) sequence showing relatively high thermostability of electrically-functional components.

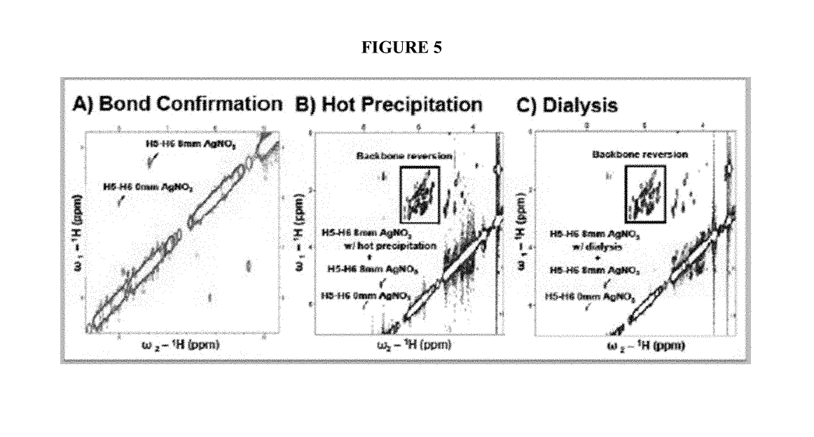

In accordance with another embodiment, the nucleic acid can provide an aromatic sheath that can shield the ions from the surrounding environment. The environment can be an aqueous, air or vacuum environment. A non-limiting example includes a continuous or discontinuous, 1-atom thick chain of silver ions surrounded by a 2 nm diameter DNA coat. In certain embodiments, correlation spectroscopy nuclear magnetic resonance (COSY-H NMR) analysis can be performed to confirm the metal ion bond site and stability under the aqueous conditions as shown in FIGS. 5A, 5B and 5C.

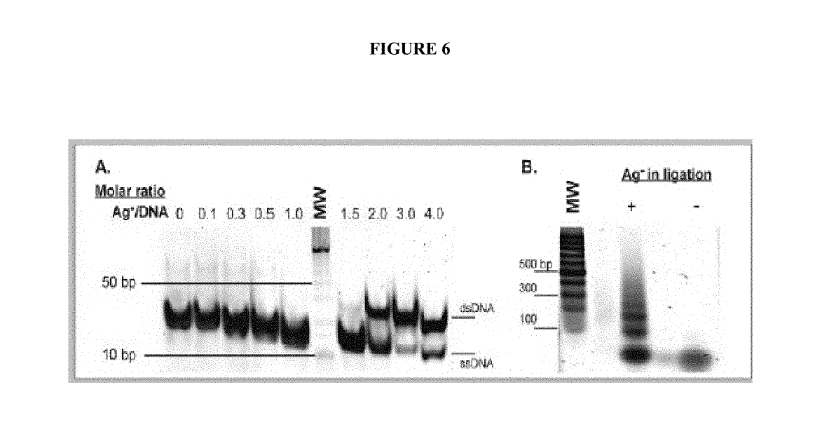

In certain embodiments, molar ratio between metal ions and mismatches in the nanostructure can be confirmed through gel electrophoresis. For example, polyacrylamide gel electrophoresis (PAGE) can be used to confirm ion dependency of duplexing in highly-mismatched oligonucleotides. FIG. 6A illustrates duplex formation can begin at 3:2 ratio, showing a near 1:1 ion-mismatch. Ligation gel in FIG. 6B illustrates up to 6-fold increase in length from 16 to −100 nm indicating that the molecules are ligase-compatible and small units can be used to form long electronic components.

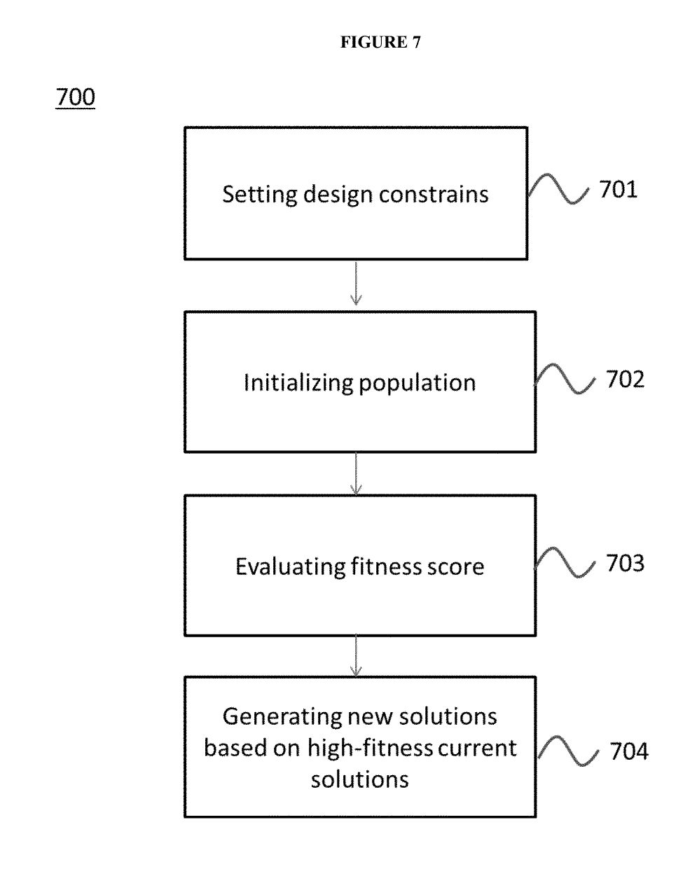

FIG. 7 is a schematic illustration of an exemplary method for designing and optimizing nucleic acid sequences in silico. In certain embodiments, a method 700 includes setting design constraints 701. For example, non-canonical base pairing, orthogonal bases, base frequency gates, base gapping (distance between iterations of a base or bases) and their equivalence rules can be added for designing nucleic acid sequences.

The method 700 can further include initializing virtual populations of sequences 702. In certain embodiments, multiple populations can be initialized with differing solution number, mutation rate, tournament size, etc. The sequences can be solutions at a given sequence length, with the given set of pairing rules. For nanostructures requiring more than one sequence per solution, each with different numbers of base pairs, the sequences can be assigned an equivalence matrix which corresponds each individual nucleic acid with the location of its logical compliment (or non-canonical compliment). The fitness rules can be overlaid in their own rule matrix to be assigned to the segment of each sequence to where they are active. This allows certain rules to apply to only small parts of a sequence, allowing heterogeneity of structure and function within a solution. The individual solutions in a population can be generated randomly or be assigned a start bias based on generalized motifs or previous solutions.

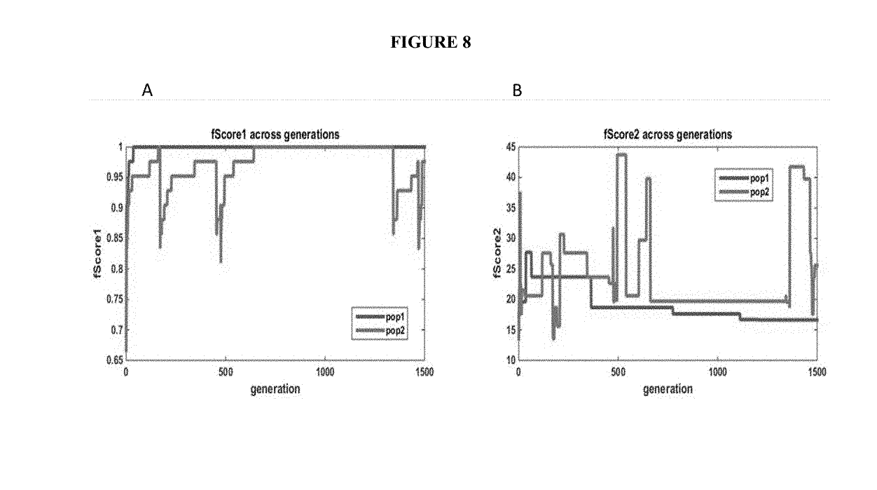

The method 700 can further include evaluating the fitness score of the sequences 703. A method of evaluating the fitness can include performing random operation to change it slightly for the next generation, and repeating in order to improve the fitness of a set of solutions. For example, a multi-objective fitness optimization can be developed to first force a population to fit a single criterion: either gapN (every nth base is cytosine—for electrical wires), or gating (% single base or pair of bases—used to deplete sequences of cytosine for assigning areas with non-conductive resistors), and then to minimize the size and frequency of unwanted secondary structures. The second criterion can use a formula to combine the size of the maximum homo- and hetero-dimers as well as the total max number of overlapping bases into a single number. The homo-dimers are unwanted configurations between copies of the same sequence. The hetero-dimers are misaligned bond confirmations between a sequence and its complement. Gating and gapping can be evaluated and compared by normalizing % completeness relative to the prescribed fitness maximum for a specific region. FIG. 8 illustrates plots of an exemplary fitness score evaluation in two populations (size 150 and 70) across 1500 generations. Population one (blue) serves as the main, stable solution space, while population two (orange) is exposed to much higher mutation rates and turnover to introduce sequence diversity into population one. The maximization of fitness score (fScore1) with the adherence to gap1 criteria is shown in FIG. 8A. FIG. 8B illustrates the minimization of fitness score (fScore2) with the thermodynamic strength of unwanted secondary structures over the generations. Population one has 60 individuals with a 0.05 mutation rate, while population two has 40 individuals with a mutations rate of 0.08 and 20% elimination bottleneck every 250 generations, where an individual is a given oligonucleotide sequence or solution. Five individuals are passed from population two to population one every 200 generations. Population one quickly maximizes fScore1 and gradually minimizes fScore2 to produce a final optimized solution at 42 bp: ACCGCTCGCACACGCCCACACACGCCGCACACTCCACCGCCG, where each C will bind to a silver ion.

The method 700 can further include generating new solutions based on high-fitness current solutions 704.

In certain embodiments, the nucleic acid sequences can be further optimized by evaluating population dynamics, subpopulations that contain or express: mating types, fitness tournaments, random mutation, extinction, recessive information and random death of solutions. The population dynamics can include alterable mutation rate, sequence exchange between separate solution pools, expression of recessive information to allow diversity of solutions even between seemingly identical solutions, etc. Further embodiments allow optimization of the equivalence matrix to promote ideal nanostructure connectivity.

In certain embodiments, the method for the optimization can iteratively optimize a solution set using novel dynamics and rules. An arbitrarily-long list of fitness scores can be used to compute the effectiveness of a particular solution. The iteration of the method is based on closed and open criteria. Closed criteria are parameters which much be attained prior to moving on to optimize a subsequent fitness score. This introduces a hierarchy within the closed rules. Selection of the order will speed or slow computation considerably. The model is able to also optimize an open fitness score, one with no prescribed threshold that is optimized indefinitely. Various parameters can be compiled into a single score and this number is minimized or maximized for the duration of the simulation, provided the closed scores have been satisfied.

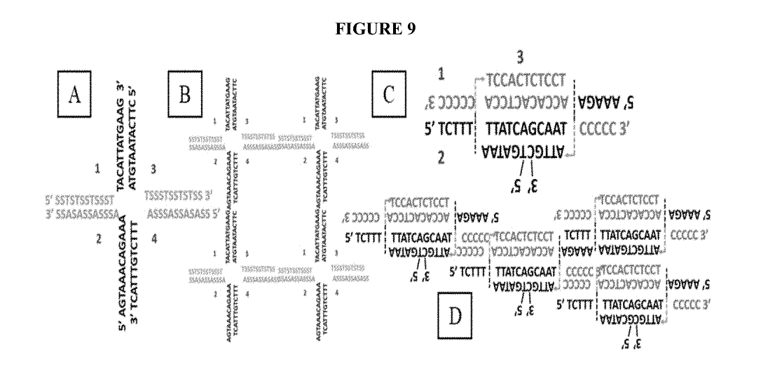

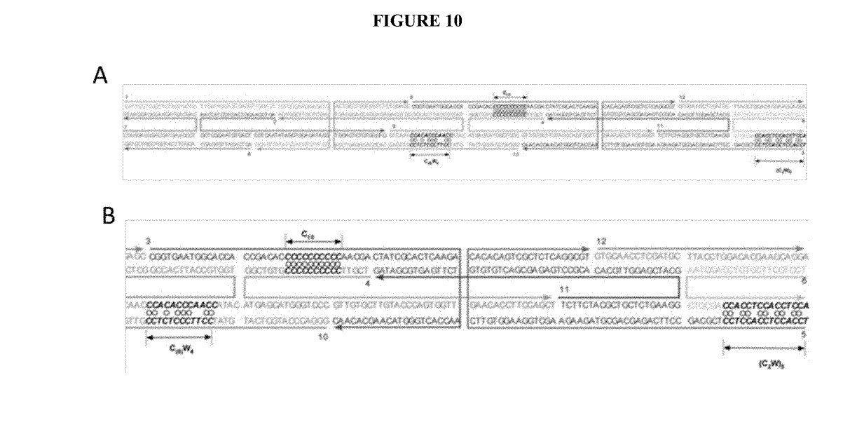

In certain embodiments, nanowire elements/nanostructures can be incorporated into any type of other nanostructures or materials to form complex electrically-functional structures. The materials can include nucleic acid materials and/or designs. For example, the nanostructure can be built into DNA lattices. The lattices can be self-assembled 1D and 2D structures. FIG. 9A illustrates 1D lattice of Holliday junctions with sticky ends. Multiple lattice units can be joined via sticky ends to form repeating lattices as shown FIG. 10B. In another embodiment, as shown in FIG. 9C, the nanostructures can be a minimal crossover unit which can weave two parallel DNA double helices together. The minimal crossover unit can be assembled linearly in a 2D tile as shown in FIG. 9D. In another embodiment, the DNA lattice can include a T-junction lattice. The DNA lattice can include at least one ion-functionalized mismatch.

In certain embodiments, the nanostructure can include at least one patterned region for metal ion insertion, or at least one core-functionalization. The nanostructure can include at least one crossover structure. In certain embodiments, the crossover structure can include at least one double-crossover structure. In another embodiments, the double-stranded DNA (or other nucleic acid) nanostructure can include at least one silver ion insertion sites.

In certain embodiments, duplex DNA five-crossover nanostructure (DFX) with silver ion binding can be utilized to construct the nanostructure. For example, a five-crossover DFX can be designed to include at least one ion-functionalized regions with at least one silver ion insertion sites (see FIGS. 10A and 10B). In accordance with another embodiment, a DNA (or other nucleic acid) nanostructure is provided that includes at least one patterned region for silver ion insertion, or core-functionalization. In certain embodiments, DFX can be built through a two part anneal process. The process can include a primary anneal at 90° C. for 48 hours and a secondary anneal at 40° C. for 72 hours. In other non-limiting embodiments, this reaction can be carried out at high temperature (90° C.) or lower temperature (40° C.) multiple times, and can be cycled for tens, hundreds or thousands of iterations.

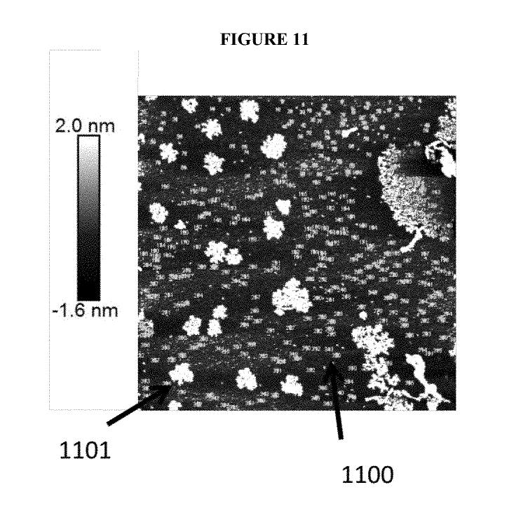

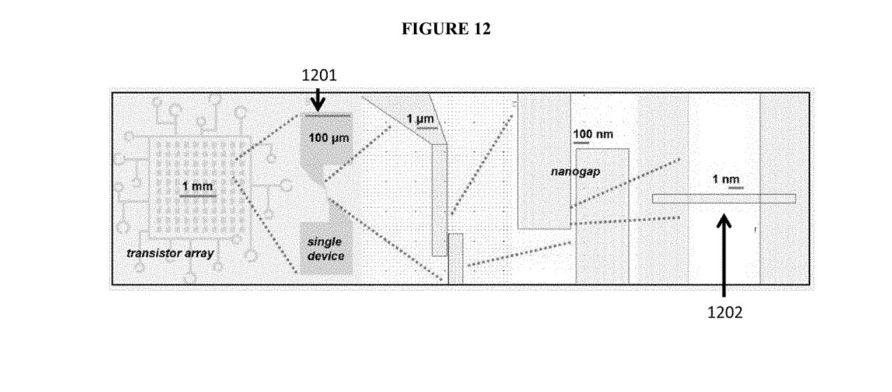

In certain embodiments, AFM image analysis can be performed to confirm the DFX fabrication by counting the number of nanostructure-sized objects. Analysis based on length and width can be performed to determine DFX formation. For example, the designed nanostructure can have a length of 60 nm, so with this information the number of probable DFX molecules can be counted through AFM image analysis. FIG. 11 illustrates nanostructure with a length of 60 nm as small dots 1100 and salt crystal 1101 that precipitated from the buffer solution as star shapes. As shown in table 1, 30% of probable DFX nanostructures fabricated in the presence of silver and only 10% in the absence.

TABLE 1
DFX image analysis for 2-part anneal process.

| | #DNA | | Yield | Probable | |
|---|---|---|---|---|---|
| | objects | #DFX- | DFX- | DFX | Highly |
| | (vary by | like | like | yield | probable |
| Silver: CC ratio | image) | objects | objects | (×66%) | (×41%) |
| 0:1 (Control) | 360 | 60 | 17% | 11% | 5% |
| 1:1 | 161 | 71 | 44% | 29% | 18% |
| 10:1 | 390 | 172 | 44% | 29% | 18% |

In certain embodiments, the silver-ion-incorporated DNA duplexes can be applied to a planar transistor system 1200. The planar transistor can be fabricated using directed assembly on high-surface-energy patterns. The use of high-resolution electron beam lithography enables these devices to serve as single-molecule transistors. FIG. 12 illustrates a design of planar transistors. Gold electrode pads 1201 are contacted externally with an applied bias voltage, allowing the electrical characterization of the single DNA or carbon nanotube (CNT) molecule 1202 assembled between the pads. Channels can be defined by trenches etched into non-fouling polyethylene glycol (PEG) to allow selective capture of molecules by the underlying OH-functionalized, silicon dioxide substrate. Devices on the ends have sites for many more molecules to allow quantification of assembly yield.

In certain embodiments, the nanostructure can comprise or include DNA (or nucleic acid) origami. Metal- or ion-binding sites can be engineered into the origami backbone. The backbone can include M13 DNA. In another embodiment, metal- or ion-binding sites can be engineered into short oligonucleotides that attach to the backbone. The short oligonucleotides can be staple strands. The origami structure can be 1D, 2D, and 3D structure.

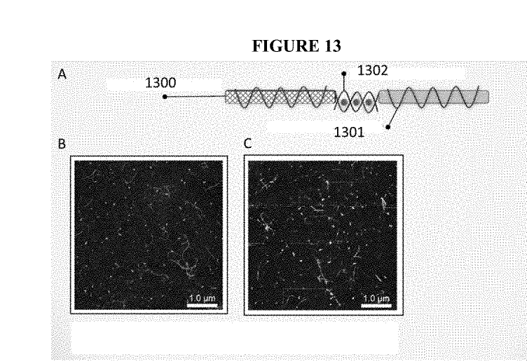

In certain embodiments, DNA nanostructures/nanowires (or networks) that include at least one metal ion-functionalized mismatch can be added or incorporated to which one other functional nanomaterial (e.g., metal nanoparticle, quantum dot, carbon nanotube, etc). For example, a carbon nanotube (CNT) can be ligated to the silver-functionalized DNA. The CNT can be used as covalently-bound leads to access 5′ and 3′ ends of DNA. As shown in FIG. 13A, two CNTs 1300 with single stand surfactant 1301 can be connected by double strand DNA 1302. Average length of CNTs can double after high-yield, solution-based coupling to amine- or guanin- functionalized dsDNA (see FIGS. 13B and 13C).

What is claimed is:

1. A method for engineering a nanostructure, comprising:
creating at least one cytosine-cytosine and/or thymine-thymine mismatch in at least one oligonucleotide sequence,
placing a metal ion into the mismatch of the oligonucleotide sequence to form an electronically functionalized nanostructure, and
inducing self-assembly of the oligonucleotide sequence into a defined structure.

2. The method of claim 1, wherein the nanostructure is incorporated to other nanomaterials.

3. The method of claim 1, wherein the placing, further comprises providing at least one metal ions to each mismatch.

4. The method of claim 1, wherein the metal ion is silver.

5. The method of claim 1, wherein the metal ion is mercury.

6. The method of claim 1, wherein the nanostructure comprises at least one fluorophore.

7. The method of claim 1, wherein the nanostructure comprises at least one biotin linker.

8. The method of claim 1, wherein the nanostructure comprises at least one fluorophore and at least one biotin linker.

9. The method of claim 1, wherein the nanostructure is designed by an optimization, wherein a method for the optimization comprises:
setting design constraints;
initializing populations;
evaluating fitness score; and
generating new solutions based on high-fitness current solutions.

10. The method of claim 9, wherein the method for the optimization analysis, further comprises evaluating population dynamics, subpopulations, fitness tournaments, random mutation, extinction, recessive traits or/and random death of solutions.

11. The method of claim 1, wherein the nanowire is a duplex DNA nanostructure having at least one core-functionalized region.

12. The method of claim 11, wherein each of the core-functionalized region comprises at least one mismatch or at least one ion-binding site.

13. The method of claim 1, wherein the nanostructure comprises a continuous or discontinuous 1-atom thick chain of the silver ions surrounded by the 2 nm diameter oligonucleotides.

14. The method of claim 1, wherein the nanostructure is assembled through annealing and/or ligation.

15. The method of claim 11, wherein the core-functionalized region, upon incorporation into at least one nanostructure, provide electrical functionalization to the nanostructure.

16. The method of claim 1, wherein the nanostructure is a DNA lattice.

17. The method of claim 1, wherein the oligonucleotide sequence comprises DNA, RNA, LNA, PNA, or XNA.
